(19) United States
(12) Patent Application Publication
(10) Pub. No.: US 2025/0295506 A1
(43) Pub. Date: Sep. 25, 2025

(54) DEGRADABLE BILE AND PANCREATIC DUCT STENT

(71) Applicant: BEIJING ADVANCED MEDICAL TECHNOLOGIES, LTD. INC., Beijing (CN)

(72) Inventors: Qinghua ZHAO, Beijing (CN); Qing LIU, Beijing (CN); Hanqing FENG, Beijing (CN); Hugh Qinghong ZHAO, Beijing (CN); Jiahong DONG, Beijing (CN); Yang YU, Beijing (CN); Ge SONG, Beijing (CN)

(21) Appl. No.: 18/726,970

(22) PCT Filed: Nov. 11, 2022
(86) PCT No.: PCT/CN2022/131301

§ 371 Date: Jul. 5, 2024

(30) Foreign Application Priority Data
Jan. 7, 2022 (CN) 202210011816.9

(51) Int. Cl. A61F 2/88

(52) U.S. Cl. CPC A61F 2/88; A61F 2230/0091; A61F 2240/001; A61F 2250/0014

(57) ABSTRACT

A degradable bile and pancreatic duct stent for dilating the narrowed lumen of a lesion, includes a guiding part, which has a guide passage through which a guide wire passes; and a stent main body, which is spirally distributed outside the guiding part in the lengthwise direction of the guiding part. The stent main body is capable of forming a spiral channel relative to the lumen, and the guide passage of the guiding part is in fluid communication with the spiral channel. The guiding part and the stent main body spirally have good bending and supporting performance, effectively drains bile, and prevent problems such as restenosis and occlusion in the bile and pancreatic duct. The guide passage is in communication with the spiral fluid channel, thus improving the drainage performance, and reducing the possibility of bile duct re-occlusion caused during the degradation process of the bile and pancreatic duct stent.

### TECHNICAL FIELD

The present disclosure generally relates to a medical device, and specifically to a degradable bile and pancreatic duct stent.

### BACKGROUND

Clinically, the main uses of gastrointestinal (GI) stents (represented by bile and pancreatic duct stents) are to treat or relieve luminal occlusion, to aid in the unidirectional flow of body fluids, and to facilitate GI tissue function and the restoration of human health.

Currently, there are two main types of bile and pancreatic duct stents in use: fixed-diameter plastic stents and self-expanding metal stents. Wherein, plastic stents are relatively inexpensive, but they need to be taken out of the body by a second operation if the stent is clogged or no longer needed; the main materials of metal stents are stainless steel, nickel-titanium alloy, platinum-iridium alloy, cobalt-chromium alloy, tantalum, titanium and so on, which are non-degradable materials. Granulation tissues will grow into the porous structure of the stent after long-term implantation, which makes the stent difficult to remove, and stents will bring some side effects if they are retained for a long period. Therefore, metallic bile duct stents are unsuitable for treating benign stenosis.

The prior art discloses a degradable bile and pancreatic duct stent, which utilizes a biodegradable material to make a base tube and constructs it into a double-helix or triple-helix structural body.

The above bile and pancreatic duct stent has the following problems:
1) The stent bending performance is unsatisfactory and the flexibility is poor, especially when the stent is longer, the stent is more difficult to pass through the part with larger curvature;
2) The inner lumen of the stent tubing is easy to be clogged, which affects the effect of bile drainage;

Based on this, the relevant technicians are committed to designing a degradable bile and pancreatic duct stent to improve the performance of the existing bile and pancreatic duct stent, and to overcome the defects existing in the prior art.

### SUMMARY OF PRESENT INVENTION

Accordingly, it is the task of present disclosure to provide a degradable bile and pancreatic duct stent, to overcome the drawbacks of the said prior art.

In order to accomplish the task, the present disclosure provides a degradable bile and pancreatic duct stent constructed to dilate the lesion lumen comprising: a guiding part constructed to have a guide passage through which a guide wire passes and a fluid flows; a stent main body spirally distributed around the guiding part along the longitudinal direction of the guiding part; the stent main body constructed to form a spiral fluid channel relative to the lumen, the guide passage of the guiding part is communicated with the spiral fluid channel.

As a preferred embodiment, the guiding part comprises at least one first spiral element, the first spiral element is spring-shaped and defines the guide passage, wherein a plurality of the first spiral elements define the guide passage in parallel with each other.

As a preferred embodiment, the stent main body comprises at least one group of spiral structures, wherein a plurality of groups of the spiral structures are distributed parallel with each other around the guiding part.

As a preferred embodiment, the guiding part comprises: a first portion, the first portion comprises at least two groups of straight elements arranged parallel with each other; a second portion, the second portion is configured in a ring shape and arranged at an end of the first portion to define the guide passage together with the first portion; the stent main body comprises at least one group of spiral structures, wherein a plurality of groups of the spiral structures are distributed parallel with each other around the guiding part along the first portion to confine the guide wire within the guide passage.

As a preferred embodiment, the guiding part is constructed as a porous structural element with said guide passage.

As a preferred embodiment, the stent main body comprises two groups of spiral structures spirally extending around the guiding part, and each group of the spiral structures comprises at least one second spiral element.

As a preferred embodiment, the spiral structure comprises two second spiral elements arranged in parallel.

As a preferred embodiment, the spiral structure comprises three spirally arranged second spiral elements.

As a preferred embodiment, the stent main body and the guiding part are made of different materials and/or the stent main body and the guiding part have different material degradation rates.

As a preferred embodiment, the degradable bile and pancreatic duct stent (1) is made by 3D printing technology.

The bile and pancreatic duct stent provided by the present disclosure provides a novel degradable bile and pancreatic duct stent with good bending performance, support performance, effective bile drainage and avoidance of restenosis and occlusion in the bile and pancreatic duct by adopting a guiding part with a guide passage and a stent main body attached to the guiding part in a spiral shape. In particular, the interconnection between the guide passage of the guiding part and the spiral fluid channel formed by the stent main body relative to the lumen improves the drainage performance of the degradable bile and pancreatic duct stent, while also reducing the possibility of the degradable bile and pancreatic duct stent causing bile duct re-occlusion during the degradation process.

Some of the other features and advantages of the present disclosure will be apparent to those skilled in the art after reading the present disclosure, and the other part will be described in the following specific embodiments in conjunction with the figures.

### BRIEF DESCRIPTION OF THE DRAWING

The embodiments of the present disclosure are described in detail below in the following figures, which show:

FIG. 1: a three-dimensional schematic diagram of an embodiment of a degradable bile and pancreatic duct stent according to present disclosure;

FIG. 2: a three-dimensional schematic diagram of a degradable bile and pancreatic duct stent with different pitches based on the embodiment of FIG. 1;

FIG. 3: a three-dimensional schematic diagram of a degradable bile and pancreatic duct stent with different pitches based on the embodiment of FIG. 1;

FIG. 4: a three-dimensional schematic diagram of another embodiment of a degradable bile and pancreatic duct stent according to present disclosure;

FIG. 5: a three-dimensional schematic diagram of another embodiment of a degradable bile and pancreatic duct stent according to present disclosure;

FIG. 6: a three-dimensional schematic diagram of a degradable bile and pancreatic duct stent with a guiding part having different numbers of bars based on the embodiment of FIG. 5;

FIG. 7: a schematic diagram of the overall structure of the degradable bile and pancreatic duct stent based on the embodiment of FIG. 1.

### REFERENCE NUMBER

1—degradable bile and pancreatic duct stent; 10—guiding part; 100—guide passage; 102—first spiral element; 104—first portion; 106—second portion; 108—barb; 12—stent main body; 120—spiral structure; 122—second spiral element

### DETAIL DESCRIPTION

Exemplary embodiments of the degradable bile and pancreatic duct stent according to present disclosure are now described in detail with reference to the figures. The figures are provided to present a plurality of embodiments of present disclosure, but the figures do not have to be drawn to the dimensions of the specific embodiments, and certain features may be enlarged, removed, or partially sectioned to better illustrate and explain the disclosure of present disclosure. Some of the components in the figures may be repositioned according to actual needs without affecting the technical effect. The phrase “in the figures” or similar terms appearing in the specification do not need to refer to all the figures or examples.

Certain directional terms used hereinafter to describe the figures, such as “inside”, “outside”, “up”, “down”, and other directional terms will be understood to have their normal meanings and to refer to those directions involved in normal viewing of the figures. Unless otherwise indicated, the directional terms described herein are substantially in accordance with conventional directions as understood by those skilled in the art.

The terms “first”, “first one”, “second”, “second one” and similar terms used in present disclosure do not denote any order, quantity, or importance, but are used to distinguish one component from others.

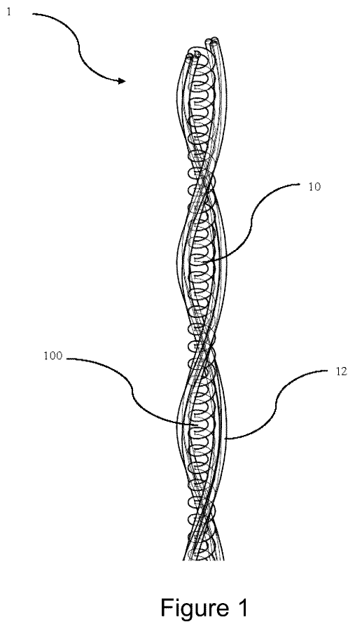

FIG. 1 is a three-dimensional schematic diagram of degradable bile and pancreatic duct stent 1 according to present disclosure. Referring to FIG. 1, the degradable bile and pancreatic duct stent 1 comprises a guiding part 10 and a stent main body 12, wherein the guiding part 10 has a guide passage 100, and a guide wire passes through the guide passage 100 so that the stent can enter a lumen of lesion in the bile and pancreatic duct by means of the guide wire. Specifically, in practical applications, for example, in the endoscopic examination, an endoscope (e.g. duodenoscope) sequentially enters into the stomach through the oral cavity and esophagus and then enters into the duodenum, and finally reaches the vicinity of the entrance of the bile duct (nipple), and a guide wire (e.g. a zebra guide wire) can reach the inside of the bile duct along the duodenoscope, and then push the bile and pancreatic duct stent provided by present disclosure into the bile duct along the zebra guide wire. The bile duct is oriented usually about 135° with respect to the right rear of the duodenoscope. The bile and pancreatic duct stent can be positioned so that its tail end can be left outside the bile duct.

Herein, the directional references “proximal” and “distal” refer to the side close to the clinician, and the side away from the clinician, and close to the examination target, respectively. The degradable bile and pancreatic duct stent 1 of present disclosure can be constructed as a structure with a gradual variation of diameter, for example, when placed in an endoscope, it has a smaller diameter on its distal side and a larger diameter on its proximal side. By this way, after the bile and pancreatic duct stent is positioned at the target position, for example, only one end is placed in the bile duct, while the other remains outside the bile duct. Such a bile and pancreatic duct stent is beneficial for the discharge of the bile duct during degradation.

The spiral element constituting the guiding part 10 of the bile and pancreatic duct stent is made by 3D printing technology, specifically, in a 3D printer, where the material is printed on a controllably rotatable metal rod (the mould of the 3D printer with a diameter of about 1 mm) using 3D technology.

In a specific embodiment, the stent main body 12 optionally comprises at least one group of spiral structures 120, preferably two groups of spiral structures 120. When there is a plurality of groups of spiral structures 120, the groups of spiral structures are arranged parallel with each other along a longitudinal direction of the guiding part 10. The stent main body is arranged on the periphery of the guiding part 10, and each group of spiral structures 120 of the stent main body may comprise at least one second spiral element 122. In other words, the stent main body 12, in particular its second spiral elements 122, is arranged in extending around the guiding part 10 spirally along the longitudinal direction of the guiding part 10. The number of second spiral elements 122 constituting each group of spiral structures 120 in the stent main body 12 may optionally be one or more. Each second spiral element 122 of the group of spiral structures 120 in the stent main body is also made by 3D printing technology, specifically, in a 3D printer, where the material is printed on a guiding part made on a controllably rotatable metal rod (the mould of the 3D printer with a diameter of about 1 mm).

When a (second) spiral member is printed on a controllably rotatable metal rod of the 3D printer (briefly described as attaching a print material to the metal rod regarded as a matrix thereof, molding it, and then demolding it to obtain the spiral element, of course, the actual printing process is more complicated, only briefly described herein to describe the structure of the spiral element), when the stent main body 12 has two groups of spiral structures 120, the two groups of spiral structures 120 extend parallel with each other (in particular, extending in a double helix manner) spirally along the longitudinal direction of the guiding part. The parallel minimum spacing between the two groups of spiral structures 120 is preferably greater than 1 mm. Considering that guiding part 10 is arranged between, for example, two groups of spiral structures 120 of the stent main body 12 (arranged to extend in the longitudinal direction of the stent main body), the distance between the two groups of spiral structures 120 of the stent main body 12 is, for example, optionally 3.4 mm or greater.

The 3D printing (3DP) mentioned herein is a rapid prototyping technology, also known as additive manufacturing. It is a technology that uses a digital model file as the basis, uses powdered metal or plastic and other bondable materials, and constructs objects by printing in a layer by layer manner. 3D printing is usually achieved using a digital technology material printer. It is often used to make moulds in the fields of mould manufacturing, industrial design, etc., and can also be used for the direct manufacturing of some products. There are already parts printed by this technology. The working principle of a 3D printer is basically the same as that of an ordinary printer, but the printing materials used are somewhat different. The “printing materials” of a 3D printer are mainly raw materials such as metal, ceramics, plastic, sand, etc. that are contained in it. After the 3D printer is connected to a computer, the “printing materials” can be stacked layer by layer through computer control, and finally the blueprint on the computer can be turned into a physical object. In other words, a 3D printer is a device that can “print” a real 3D object. The spiral structure 120 of the stent main body 12 forms an open channel with respect to the guiding part 10 when it is wound in a spiral way on the guiding part 10, and when the stent main body 12 is arranged within the lumen of the lesion of the bile and pancreatic duct, the open channel forms a spiral channel together with the lumen of the lesion. The flow in guide passage 100 of the guiding part 10 is fluidly communicated to the spiral channel formed by the spiral structure 120 of the stent main body 12. The person skilled in the art understands that when the stent main body 12 optionally has only one group of spiral structure 120 (not shown in the figures), the stent main body 12, in particular the second spiral element 122 of the spiral structures 120, has a pitch greater than the pitch of the first spiral element 102 forming the guide passage 100.

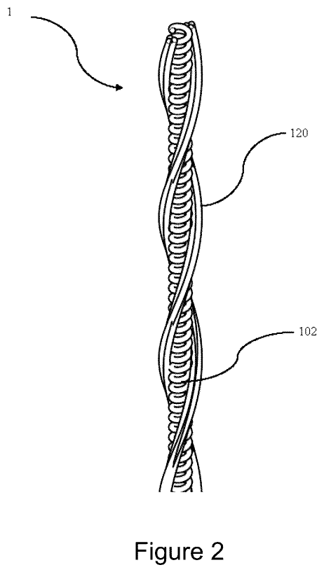

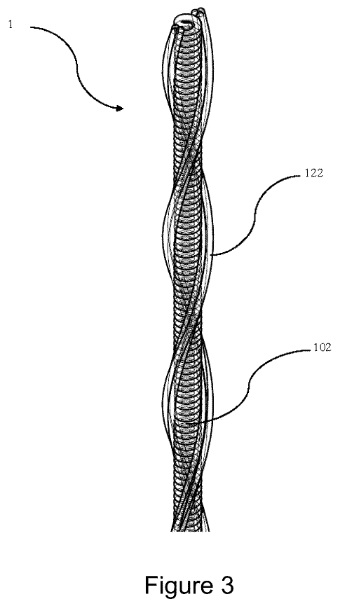

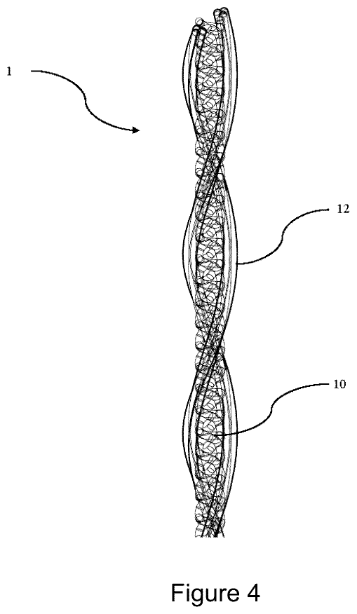

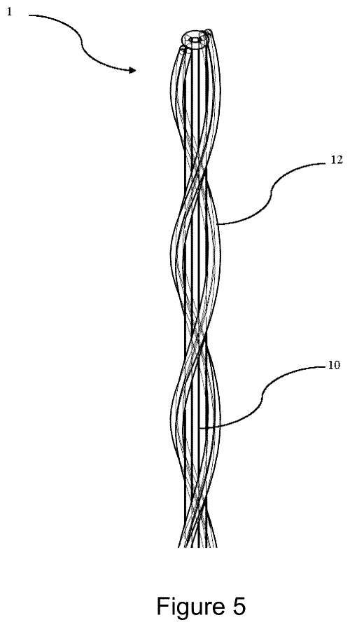

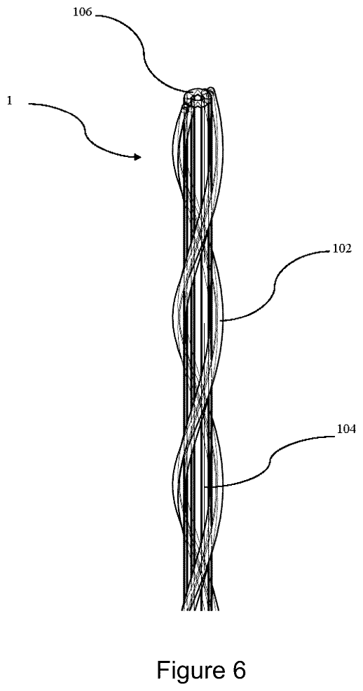

In FIGS. 1 to 3, the guiding part 10 each comprises a first spiral element 102, the difference among the embodiments shown in each figure is the difference of the pitch among the individual first spiral element 102; in FIG. 4, the guiding part 10 comprises two first spiral element 102, wherein the two first spiral element 102 are arranged parallel with each other; and in FIGS. 5 to 6, the guiding part 10 each comprises a two-part structure, wherein the first portion is a linear element and the second portion is a ring structure (which will be described in detail below and will not be repeated here), the difference among the embodiments shown in the figures is that the first portion 104 has two or more groups of linear elements, wherein each group has at least one linear element. Of course, it may be understood by the person skilled in the art, inspired by the technical solution of present disclosure, that the number of the first spiral element 102 of the guiding part 10 or the number of groups of the linear elements as the first portion may be adjusted as required, and such modifications and variations fall within the scope of present disclosure.

Taking the guiding part 10 constructed as the structure of the embodiments shown in FIGS. 1 to 4 as an example, the pitch ratio between the stent main body 12, in particular the second spiral element 122 of the spiral structure 120 thereof, and the first spiral element 102 constituting as the guiding part 10, provided that they each fulfill their function for example, can be selectively preferably set in the range of 1-1200, preferably, the pitch ratio can be 6-60, more preferably 12.

Additionally, the stent main body 12, particularly the second spiral element 122 of the spiral structure 120 thereof, may have a different spiral direction than the spiral direction of the first spiral element 102 constructing as the guiding part 10. The pitch ratio and spiral direction of the stent main body 12 and the guiding part 10 should be chosen in such a way as to satisfy the required flexibility/curvature of the bile and pancreatic duct stent for traveling in a curved path, for example optionally from 0.005 to 0.1 N/mm.

As previously mentioned, when the stent main body 12, in particular, its spiral structure 120 is in two groups, the two groups of spiral structures 120 of the stent main body 12 are spirally wound (i.e. parallel with each other) on the guiding part 10 in a double helix manner, see the embodiment of FIGS. 1 to 4, and the pitch of the stent main body 12, in particular, the second spiral element 122 of the spiral structure 120 thereof is clearly greater than the pitch of the guiding part 10. Preferably, the pitch ratio between them may for example be 2-1200, preferably 12-120, more preferably 24.

When the bile and pancreatic duct stent 1 is placed in a lesion lumen of the bile and pancreatic duct, bile flows through the lesion lumen through the guide passage 100 of the guiding part 10 on the one hand, and also through the lesion lumen through the spiral channel formed by the stent main body 12 with respect to the lumen on the other hand, and because the guide passage 100 and the spiral channel are in fluid communication with each other, bile may also flow to the spiral channel through the guide passage 100 or flow into the guide passage through the spiral channel. Since the bile circulating in the lesion bile and pancreatic duct may carry bile sludge which is prone to cause the bile and pancreatic duct stent to be clogged and dead cells which are prone to hang on the wall, the bile and pancreatic duct stent provided by present disclosure is capable of effectively avoiding the occurrence of these problems because of the structure mentioned above, making the flow of the bile more unimpeded, reducing the situation of the bile sludge being clogged and the cells hanging on the wall, and effectively improving the efficiency of the flow of the bile in the bile and pancreatic duct.

In order to further illustrate the function and effect of the bile and pancreatic duct stent provided by present disclosure compared to the bile and pancreatic duct stent of the prior art (Archimedes stent as an example), the applicant performed the following comparative experiment. In order to simulate the drainage effect of the bile and pancreatic duct stent used for drainage in the bile duct in case of occlusion of the inner wall of the bile duct, the applicant performed the following operation.
(I) The two types of bile and pancreatic duct stents to be compared were selected, and a sealing film was used to wrap the outer surface of the middle part of the bile and pancreatic duct stents to obstruct the external fluid channel formed on the outer surface of the bile and pancreatic duct stents, then the treated bile and pancreatic duct stents were placed into silicone tubes with an inner diameter of 3.00 mm to simulate the occurrence of the occlusion of the middle part of the outer portion of the bile and pancreatic duct stents in the bile ducts;
(II) re-selecting two fresh bile and pancreatic duct stents mentioned above, repeating the operation of using a sealing film in the middle part of the bile and pancreatic duct stent to obstruct the outer surface thereof in (I), and then using a fiber thread (e.g., a PDO thread) to obstruct the inner lumens of both of the bile and pancreatic duct stents and then putting the treated bile and pancreatic duct stents into a silicone tube with an inner diameter of 3.00 mm to simulate the bile duct environment where the bile impurities block the inner lumen of the bile and pancreatic duct stent.

The bile and pancreatic duct stent of the present disclosure was compared to the Archimedes stent by measuring the volume of water passing through the silicone tubing over a period of 1 minute at a hydraulic pressure of 0.66 kPa. The results showed (see table below) that (I) when the outer middle portion of the bile and pancreatic duct stent was clogged, the fluid flow rate of the bile and pancreatic duct stent provided by the present disclosure was about twice as high as that of the Archimedes stent (17.0 ml/min vs. 9.8 ml/min); and (II) when both the outer middle portion of the bile and pancreatic duct stent and the inner lumen of the bile and pancreatic duct stent were clogged, the fluid flow rate of the bile and pancreatic duct stent provided by the present disclosure could still be maintained at 16.7 ml/min, while the liquid flow rate of the Archimedes stent was zero.

It is thus shown that in the drainage experiments of simulation of the bile and pancreatic duct stent used for drainage in the bile duct under the case of occlusion of the inner wall of the bile duct, the stent of present disclosure is significantly better than the comparative stent in terms of drainage effect.

| | Occlusion simulation | | | | | | | |
|---|---|---|---|---|---|---|---|---|
| | Occlusion of the outer middle | | | | Occlusion of the outer middle of | | | |
| | of the stent (I) quantity of | | | | the stent and the internal drainage | | | |
| | liquid flow (ml/min) | | | | (II) quantity of liquid flow (ml/min) | | | |
| | Experi- | Experi- | Experi- | Average | Experi- | Experi- | Experi- | Average |
| | ment 1 | ment 2 | ment 3 | (ml/min) | ment 1 | ment 2 | ment 3 | (ml/min) |
| Stent of | 17.0 | 17.0 | 16.9 | 17.0 | 17.0 | 16.8 | 16.3 | 16.7 |
| invention | | | | | | | | |
| Archimedes | 10.5 | 9.8 | 9.0 | 9.8 | 0 | 0 | 0 | 0 |
| stent | | | | | | | | |

The guiding part 10 and the stent main body 12 may be made of different materials (e.g., degradable metals and degradable polymers) and/or polymers with different degradation rates, such as PLA (polylactic acid), PCL (polycaprolactone), PLGA (poly (lactic-co-glycolic) acid), PDO (Polydioxanone), PDX (Polydioxanone), PLC (which is a copolymer of PLLA and PCL with the molecular formula [(C6H8O4)x(C6H10O2)y]n, L-lactic caprolactone copolymer), PBAT (copolymer of butylene glycol adipate and butylene terephthalate), etc.

The guiding part 10 and the stent main body 12 can be degraded after the bile and pancreatic duct stent had been applied to the lesion lumen for a certain period, the degradable bile and pancreatic duct stent can avoid the long-term chronic adverse effects attributable to the permanent metal stent. In this regard, the difference in degradation rate between the guiding part 10 and the stent main body 12 of the bile and pancreatic duct stent 1 provided by present disclosure can meet the needs of the bile and pancreatic duct stent degradation on the one hand, it can avoid the bile and pancreatic duct stent from collapsing due to the insufficient support during the period of degradation on the other hand, or even cause the bile and pancreatic duct to have secondary stenosis and other problems.

Referring to FIGS. 1 to 3, the guiding part 10 may be formed by a first spiral element 102 made from one or more of the materials described above wound in a spiral shape (e.g. spring-like, the pitch of which may be designed according to the different requirements, e.g. the pitch of which may be optionally located between 0.1 mm and 10 mm, thereby providing guiding part 10 with different passage rates) having a substantially centered guide passage 100 formed in the longitudinal direction of the spiral extension. The guide passage 100 is formed in a longitudinal direction for navigating the guide wire through it and may guide the flow of bile through the bile and pancreatic duct stent 1.

As shown in FIG. 4, the guiding part 10 may also be formed by two first spiral elements 102 made from one or more of the materials described above arranged parallel with each other (the structure of which may be referred to as a double helix). The guide passage 100 of the guiding part 10 is defined along the longitudinal direction of the two first spiral elements 102.

The guiding part 10 may also be a porous structural element made from one or more of the materials described above with a number of holes. The porous structural element may optionally be made of individual tubing and the communication of the lumen to the outside may be achieved by punching holes in the tube, the perforated area in the tube being, for example, 0-75% of the surface area of the tube.

For the guiding part 10 comprised of the first spiral element 102 provided by present disclosure, the flexibility (i.e., the ability to bend with different forces) of the entire bile and pancreatic duct stent is enhanced, thereby facilitating the passage of this bile and pancreatic duct stent through the bending site to reach the site of the lesion, and solving the difficulty for the stent that exists in the prior art when passing through the bending site with too large curvature. In addition, such a guiding part 10 can guide the bile flow into the spiral channel of the stent main body 12 in a plurality of directions, thereby avoiding bile sludge deposition and possible stent clogging caused by the accumulation of dead cells hanging on the wall.

Alternatively, as shown in FIGS. 5 to 6, the guiding part 10 may comprise two portions, i.e. a first portion 104 and a second portion 106. The first portion 104 comprises at least two groups of linear elements arranged parallel with each other, and the second portion 106 is constructed in the form of an annulus, the second portion 106 is arranged at the end of the first portion 104, and the linear elements that are parallel with each other are fixed by being attached to the annular second portion 106 at the end, so that the first portion and the second portion together define a guide passage 100 for the guide wire to get through, and also support each other with the stent main body 12 that is spirally (e.g. in a double-helix manner) wrapped around the outside of the guiding part 10.

In this embodiment, the guiding part 10 provides a traveling space for the guide wire in the axial direction through an axial space defined by two groups of linear elements (each group having, for example, one linear element) of the first portion 104 attached to the second portion 106, while the stent main body (with two groups of spiral structures of the stent main body 12, for example, but not limited to this) spirally wound thereon further limits the traveling space of the guide wire in the radial direction of the guiding part 10 (this is mainly achieved by rationally designing and adjusting the stent main body 12, especially the pitch of the spiral structure), so as to avoid the guide wire passing through the guiding part 10 (in the case of meeting the requirement of flexibility when passing through the curved path in the body) from escaping out of the degradable bile and pancreatic duct stent through the spiral gap of the stent main body 12.

As described above, the difference between FIG. 5 and FIG. 6 is that the first portions defining the guiding part have two or more groups of linear elements. In FIG. 5, the first portion 104 (two groups of linear elements parallel with each other, the number of the linear elements in each group can be adjusted as required, as the figure shows, the number of linear elements is single one) achieves a certain degree of support and guidance together with the stent main body 12 by spaced apart from each other in parallel on the circumferential surface of the annular second portion 106; however, in FIG. 6, the first portion 104 (optionally more than two groups of parallel linear elements, the number of linear members in each group can be adjusted as required, as the figure shows, the number of linear elements is single one) achieves a certain degree of support and guidance together with the stent main body 12 by being paralleled spaced apart from each other in the axial direction of the annular second portion 106.

In the embodiment of FIGS. 5 and 6, the number of second spiral elements 122 constituting as the stent main body 12 is optionally 2. When the number of second spiral elements 122 is more than or equal to 3, the stent main body 12 shows a stabilized state that is not beneficial for bending, which is not beneficial for making an adaptable deformation (bending) for the degradable bile and pancreatic duct stent when facing a complex curved path.

In one embodiment, the mould (i.e. a controllably rotatable metal rod) used for the degradable bile and pancreatic duct stent of the present disclosure obtained by the 3D printing machine typically has a diameter of 1 mm. The diameter of the guide wire is 0.89 mm. As described above, the minimum pitch between the two groups of spiral structures of the stent main body 12 that extend spirally in parallel with each other along the guiding part 10 (in particular, extending in a double helix manner) is preferably greater than 1 mm. The guide wire can travel in the space defined by the guiding part 10 and the stent main body 12, the guide wire generally travels close to the inner wall of the bile and pancreatic duct stent instead of in the middle of the bile and pancreatic duct stent, and meanwhile the bending of the guide wire affects and adjusts the form of the bile and pancreatic duct stent, so as to achieve the bile and pancreatic duct stent bend in a substantially synchronous manner with the guide wire.

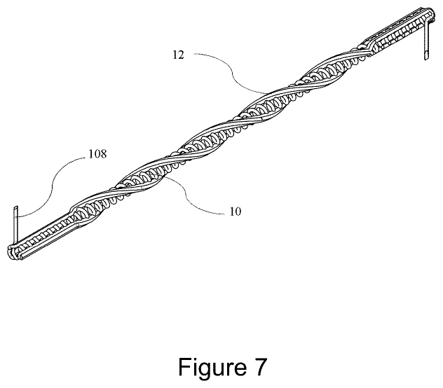

FIG. 7 shows a schematic diagram of the overall structure of the degradable bile and pancreatic duct stent based on the embodiment shown in FIG. 1. It further shows an end structure located at both ends of a body portion (referring to a portion comprising a guiding part and a stent main body) of the bile and pancreatic duct stent. At the end of the bile and pancreatic duct stent, the end structure is mainly formed by the extension structure of the stent main body 12. In the end structure of the bile and pancreatic duct stent, for example, at least one second spiral element of a group of spiral structures of the stent main body protrudes away from the axial direction of this end structure and toward the radial direction to form a positioning element (e.g. barb 108) of the bile and pancreatic duct stent, the barb facilitates in the positioning of the bile and pancreatic duct stent at a predetermined location within the body.

For a bile and pancreatic duct stent with a diameter of 2.0 mm, the barb 108 is angled at an angle of 39°-60° with respect to the axial direction of the bile and pancreatic duct stent (in particular body portion thereof); for a bile and pancreatic duct stent with a diameter of 2.6 mm, the barb 108 is angled at an angle of 43°-60° with respect to the axial direction of the bile and pancreatic duct stent (in particular the body portion thereof); for a bile and pancreatic duct stent with a diameter of 3.4 mm, the barb 108 is angled at an angle of 45°-60° with respect to the axial direction of the bile and pancreatic duct stent (in particular the body portion thereof). Alternatively, for the bile and pancreatic duct stents of different diameters described above, the barbs 108 are preferably angled at 45°-60° with respect to the axial direction of the bile and pancreatic duct stent (in particular the body portion thereof).

In addition, based on the material selection of the guiding part 10 and the stent main body 12, and the design of the holes of the guiding part 10 for bile to pass through, the bile and pancreatic duct stent provided by present disclosure can, on the one hand, realize adjust its degradation rate purposely, on the other hand, can pre-determine the location of the breakpoint of the bile and pancreatic duct stent, so as to effectively control the size of the degradation fragments of the bile and pancreatic duct stent, and to avoid problems such as additional bile duct re-occlusion or collapse of the bile and pancreatic duct stent due to the size of the degradation fragments.

As described above, the stent main body 12 is attached to a surface of the guiding part 10. The stent main body 12 comprises at least two groups of spiral structures 120 spirally distributed around the longitudinal direction of the guiding part 10 on the outer side of the guide cavity 10, wherein each group of spiral structures 120 comprises at least one spiral element 122 made of one or more of the materials described above.

As an alternative embodiment, each group of spiral structures 120 of the stent main body 12 may also comprise two second spiral element 122, wherein the two second spiral elements 122 are, for example, arranged side by side.

As an alternative embodiment, as shown in FIGS. 1 to 6, each group of spiral structures 120 of the stent main body 12 may also comprise three second spiral elements 122.

The above configuration of the stent main body 12, in particular the spiral structures 120, facilitates in increasing the structural strength of the stent main body 12 in order to enhance the structural stability provided by the stent main body 12 to the guiding part 10 around which it is wound, as well as to enhance the support of the lesion lumen.

In addition, the bile and pancreatic duct stent shown in FIGS. 5, 1, and 4 has progressively enhanced structural strength due to the different structure of its guiding part, and the increase in structural strength is mainly reflected by the gradual decrease in the area of the internal guiding space defined by the guiding part that is in communication with the outside.

The number of spiral and/or linear elements of the bile and pancreatic duct stent is related to the diameter of the bile duct (related to the environment in which the bile and pancreatic duct stent is applied). The diameter of this bile and pancreatic duct stent needs to be enlarged in the case of abnormal bile ducts, such as bile duct hypertrophy (the specifications of each bile and pancreatic duct stent can be different according to the differences in their applied environment), and the enlargement of the diameter of the bile and pancreatic duct stent can be realized by using the increase in the number or the height of the second spiral element, thus adjusting the distance of the outer wall of the bile and pancreatic duct stent from the inner wall of the bile duct so as to facilitate the barb to securely position the bile and pancreatic duct stent within the bile duct.

The degradable bile and pancreatic duct stent 1 with the guiding part 10 and the stent main body 12 made by one or more of the materials described above can be printed in a single pass using a four-axis 3D printer. Thus, the manufacturing process of the degradable bile and pancreatic duct stent 1 produced is simplified, the manufacturing cost is reduced, and the manufacturing efficiency is improved.

The bile and pancreatic duct stent described above provided by the present disclosure provides a new type of degradable bile and pancreatic duct stent with good bending performance, support performance, and effective bile drainage and avoiding restenosis and occlusion in the bile and pancreatic duct by adopting the ingenious and reasonable structural design of the guide passage with a guiding part and the stent main which is attached to the guiding part spirally. In particular, the communication between the guide passage and the spiral fluid channel improves the drainage performance of the degradable bile and pancreatic duct stent and also reduces the possibility of bile duct re-occlusion caused by the degradable bile and pancreatic duct stent during the degradation process.

While present disclosure is described in detail using only a limited number of embodiments, it should be readily understood that present disclosure is not limited to such embodiments as disclosed. Instead, present disclosure may be modified by incorporating any number of previously described variations, alterations, substitutions, or equivalent devices, but this is equivalent to the spirit and scope of present disclosure. Furthermore, while various embodiments of the present disclosure have been described, it is understood that aspects of the present disclosure may include only some of the embodiments. Accordingly, the present disclosure is not considered to be limited by the description above, but only by the appended claims.

1. A degradable bile and pancreatic duct stent (1) for dilating the narrowed lumen of a lesion, the duct stent comprising:
a guiding part (10) configured to have a guide passage (100) through which a guide wire passes and a fluid flows; and
a stent main body (12) spirally distributed around the guiding part (10) along the longitudinal direction of the guiding part (10);
wherein the stent main body (12) is configured to form a spiral fluid channel relative to the lumen, and the guide passage (100) of the guiding part (10) is in communication with the spiral fluid channel.

2. The degradable bile and pancreatic duct stent (1) according to claim 1, wherein the guiding part (10) comprises at least one first spiral element (102), the first spiral element (102) being spring-shaped and defines the guide passage (100), wherein a plurality of the first spiral elements (102) define the guide passage (100) in parallel with each other.

3. The degradable bile and pancreatic duct stent (1) according to claim 1, wherein the stent main body (12) comprises at least one group of spiral structures (120), wherein a plurality of groups of the spiral structures (120) are distributed parallel with each other around the guiding part (10).

4. The degradable bile and pancreatic duct stent (1) according to claim 1, wherein the guiding part (10) comprises:
a first portion (104), the first portion (104) comprising at least two groups of straight elements arranged parallel with each other;
a second portion (106), the second portion (106) being configured in a ring shape and arranged at an end of the first portion (104) to define the guide passage (100) together with the first portion (104); and
the stent main body (12) comprises at least one group of spiral structures (120), wherein a plurality of groups of the spiral structures (120) are distributed parallel with each other around the guiding part (10) along the first portion (104) to confine the guide wire within the guide passage (100).

5. The degradable bile and pancreatic duct stent (1) according to claim 1, wherein the guiding part (10) is constructed as a porous structural element with said guide passage (100).

6. The degradable bile and pancreatic duct stent (1) according to claim 1, wherein the stent main body (12) comprises two groups of spiral structures (120) spirally extending around the guiding part (10), and each group of the spiral structures (120) comprises at least one second spiral element (122).

7. The degradable bile and pancreatic duct stent (1) according to claim 6, wherein the spiral structure comprises two second spiral elements (122) arranged in parallel.

8. The degradable bile and pancreatic duct stent (1) according to claim 6, wherein the spiral structure comprises three spirally arranged second spiral elements (122).

9. The degradable bile and pancreatic duct stent (1) according to claim 1, wherein the stent main body (12) and the guiding part (10) are made of different materials and/or the stent main body (12) and the guiding part (10) have different material degradation rates.

10. The degradable bile and pancreatic duct stent (1) according to claim 9 wherein the degradable bile and pancreatic duct stent (1) is made by 3D printing technology.

11. The degradable bile and pancreatic duct stent (1) according to claim 2, wherein the stent main body (12) comprises at least one group of spiral structures (120), wherein a plurality of groups of the spiral structures (120) are distributed parallel with each other around the guiding part (10).
